(19) United States
(12) Patent Application Publication
(10) Pub. No.: US 2025/0009494 A1
(43) Pub. Date: Jan. 9, 2025

(54) URETHRAL CUFF FOR USE IN ARTIFICIAL URINARY SPHINCTERS

(71) Applicant: Huseyin LULECI, Istanbul (TR)

(72) Inventors: Huseyin LULECI, Istanbul (TR); Ahmet Melih LULECI, Istanbul (TR)

(73) Assignee: Huseyin LULECI, Istanbul (TR)

(21) Appl. No.: 18/711,639

(22) PCT Filed: Nov. 30, 2021
(86) PCT No.: PCT/TR2021/051319

§ 371 Date: May 20, 2024

(51) Int. Cl. A61F 2/00; A61F 2/48

(52) U.S. Cl. CPC A61F 2/004; A61F 2/484; A61F 2220/0033

(57) ABSTRACT

A urethral cuff for use in an artificial urinary sphincter is provided. The urethral cuff has a backsheet forming the body of the urethral cuff. The backsheet is composed of a first portion and a second portion wherein the first portion has a first inflatable cushion and a second inflatable cushion which are separated from each other by a gap and the second portion has a third inflatable cushion. The third inflatable cushion is located such that it corresponds on top of said gap when the second portion is folded on top of the first portion such that when the urethral cuff is implanted around the urethra of a patient and all three inflatable cushions are inflated, a length of the urethra is flattened among the inflatable cushions resulting in occlusion of the urethra.

### CROSS REFERENCE TO THE RELATED APPLICATIONS

This application is the national phase entry of International Application No. PCT/TR2021/051319, filed on Nov. 30, 2021, the entire contents of which are incorporated herein by reference.

### TECHNICAL FIELD

The present invention relates to an improved urethral cuff for use in artificial urinary sphincters (AUS) for treating incontinence.

### BACKGROUND

A biological urinary sphincter prevents urinary flow via mucosal coaptation, compression and pressure transmission. On the other hand, an artificial urinary sphincter mimics the biological urinary sphincter by providing a competent bladder outlet during urinary storage and an open unobstructed outlet to permit voluntary urination.

A known treatment for some cases of incontinence is to provide a patient with a mechanism to occlude the affected excretory body passage. These mechanisms are typically surgically implanted within the patient's body and are adapted to be operable by the patient to selectively open and occlude the body passage. Inflatable artificial sphincters are well known devices in the state of the art. Inflatable sphincters typically include an inflatable cuff for surrounding the passage to be occluded. Usually, a pump cooperatively associated with a fluid reservoir is utilized to transfer fluid into and out of the cuff. As fluid is transferred into the cuff, the cuff inflates and occludes the circumscribed body passage.

Artificial urinary sphincters (AUS) known in the state of the art consist of three major parts, namely a fluid reservoir, a cuff and a pump which is usually designated as the control mechanism of the AUS. The pump can be placed in a man's scrotum. It can also be placed underneath the skin in a woman's lower belly, labia or leg. Two conduit tubes connect all three major parts to each other. Use of an extra element, in particular of a conduit tube, increases the implantation time, complexity of the surgery and most importantly, the infection risk of a patient after implantation within the body.

Many conventional sphincteric mechanisms, namely cuffs, are characterized by a circumferential occlusion geometry. However, these cuffs configured to apply circumferential occlusive pressures fail to efficiently occlude the excretory body passage when a sudden pressure increase occurs in the patient's abdomen. A sudden pressure increase may occur when, for instance, the patient laughs, coughs or is burst into laughter and also by way of certain physical movements such as bending the upper body down or when lifting a weight. In such cases, the normal pressure formed in the inflatable cuff may fail to effectively occlude the excretory body passage and excreted fluid which already accumulated behind the cuff or in the bladder may unintentionally leak outside the patient's body. In addition, it is a known fact that circumferential cuff applications increase the susceptibility of the urethra to atrophy or erosion.

U.S. Pat. No. 4,584,990A, for instance, draws attention to the inefficacies of sphincter structures that apply circumferential occlusive pressure and aims to develop a structure that eliminates these disadvantages to provide an efficient continence for the patient. Accordingly, the proposed occlusion cuff includes a pair of oppositely disposed, inflatable patient-activated and physician-controlled chambers. A fluid medium is selectively supplied to one or both of the chambers for causing expansions thereof and an increase in the occlusive pressures applied to the lumen. It mentions that the oppositely disposed chambers apply a minimal diametric occlusive pressure to the lumen to more effectively and efficiently achieve coaptation with a reduced possibility of atrophy, erosion and/or ischemia. However, the two-chamber mechanism proposed here applies a combined pressure in such a way as to shrink a radially narrow area of the lumen. Since the occlusive pressure remains in a single plane, there is still a risk that the fluid passage cannot be completely blocked. More importantly, a limited area of occlusion can also cause excessive compression of the capillaries in the lumen and thus resulting in damage to the living tissue. Moreover, this mechanism requires both the patient and a physician interference, therefore it needs to be simplified in order to avoid complexity and to enhance patient compliance. It also needs to be improved to ensure the efficacity in preventing incontinence.

Conventional urethral cuffs known in the state of the art pressurize the urethral canal circumferentially and continence can be established only when a high pressure is applied on the periphery of the urethra. The high peripheric pressure, however, is harmful to the urethra since the urethral canal is a living tissue and the high pressure also results in occlusion of the capillary vessels within the urethra at the plane where peripheric pressure is applied. There are reported cases where the urethra of the patient was physically harmed and was partially torn due to the high local pressure which was circumferentially applied.

### SUMMARY

Primary object of the present invention is to provide a new urethral cuff which eliminates the drawbacks of the existing cuffs uses in artificial urinary sphincters.

In particular, an object of the present invention is to provide a new occluding mechanism for a urethral cuff which improves the efficacity in overcoming incontinence without damaging the capillaries in the urethra and the corresponding tissue.

A further object of the present invention is to provide a urethral cuff providing enhanced patient compliance and enhanced incontinence security.

A further object of the present invention is to provide a simplified urethral cuff which is easy to manufacture.

A further object of the present invention is to provide a urethral cuff which allows insertion of an endoscope or a resectoscope while deflated.

A final object of the present invention is to provide a simplified urethral cuff which is easy to implant.

### BRIEF DESCRIPTION OF THE DRAWINGS

The figures whose brief explanations are herewith provided are solely intended for providing a better understanding of the present invention and are as such not intended to define the scope of protection or the context in which said scope is interpreted in the absence of the description.

FIG. 1 shows a perspective view of an artificial urinary sphincter of the prior art.

FIG. 2 shows the top view of the urethral cuff according to the present invention, in the inflated and open state of the urethral cuff.

FIG. 3 shows the top view of the urethral cuff given in FIG. 2, in the deflated and open state of the urethral cuff.

FIG. 4 shows the front view of a urethral cuff according to the present invention, holding a urethra, in the inflated and folded state of the urethral cuff.

FIG. 5 shows a front cross-sectional view of the urethral cuff given in FIG. 4, holding a urethra, in the inflated and folded state of the urethral cuff.

FIG. 6 shows the side view of the urethral cuff according to the present invention, holding a urethra, holding a urethra, in the inflated and folded state of the urethral cuff.

FIG. 7 shows the side view of the urethral cuff given in FIG. 6, holding a urethra, in the deflated and folded state of the urethral cuff.

FIG. 8 shows a top perspective view of the urethral cuff according to the present invention, in the inflated and open state of the urethral cuff.

FIG. 9 shows a top perspective view of the urethral cuff given in FIG. 8, in the deflated and open state of the urethral cuff FIG. 10 shows a top perspective view of the urethral cuff according to the present invention, holding a urethra, in the inflated and folded state of the urethral cuff.

FIG. 11 shows a top perspective view of the urethral cuff given in FIG. 10, holding a urethra, in the deflated and folded state of the urethral cuff.

FIG. 12 shows a top view of the urethral cuff according to the present invention, holding a urethra, in the inflated and folded state of the urethral cuff.

FIG. 13 shows a top view of the urethral cuff given in FIG. 12, holding a urethra, in the deflated and folded state of the urethral cuff.

FIG. 14 shows a bottom perspective view of an embodiment of the urethral cuff according to the present invention in the inflated and folded state of the urethral cuff.

FIG. 15 shows a bottom perspective view of another embodiment of the urethral cuff according to the present invention in the inflated and folded state of the urethral cuff.

### DETAILED DESCRIPTION OF THE EMBODIMENTS

The list of reference numerals used in the appended drawings is as follows;
1. Urethral cuff
2. Backsheet
   21. First portion
   22. Second portion
3. Gap
4. Recess
5. Inlet
   51. Protrusion
   52. Inlet hole
6. Folding section
   61. Conduit
7. Pull tab
   71. Connection hole
8. Reinforcement element
10. First inflatable cushion
20. Second inflatable cushion
30. Third inflatable cushion
40. Fourth inflatable cushion
100. Urethra
200. Length
300. Tube
400. Pump
500. Fluid reservoir
600. Cuff of the prior art

Objects of the present invention are achieved by the features of Claim 1 in which a urethral cuff (1) for use in an artificial urinary sphincter is disclosed. The urethral cuff (1) has a backsheet (2) forming the body of the urethral cuff (1) and composed of a first portion (21) and a second portion (22). The first portion (21) has a first inflatable cushion (10) and a second inflatable cushion (20) which are separated from each other by a gap (3). The second portion (22) has a third inflatable cushion (30) which is located such that said third inflatable cushion (30) corresponds on top of said gap (3) when the second portion (22) is folded on top of the first portion (21) such that when the urethral cuff (1) is implanted around the urethra (100) of a patient and all three inflatable cushions (10, 20, 30) are inflated, a length (200) of the urethra (100) is flattened among the inflatable cushions (10, 20, 30) resulting in occlusion of the urethra (100).

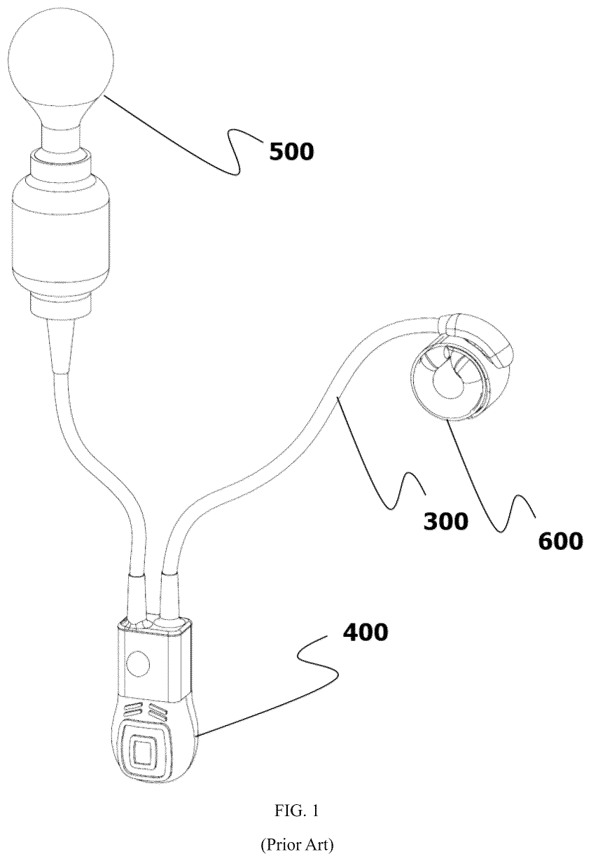

An artificial urinary sphincter of the prior art comprising a pump (400), a fluid reservoir (500) and a cuff (600) is given in FIG. 1. The cuff (600) shown here is configured to diametrically shrink and occlude the urethra unlike the present invention in which a urethral cuff (1) occluding the urethra (100) by flattening a length (200) of the urethra (100) is proposed.

The advantage of the occluding mechanism according to the present invention is that since the urethra (100) is compressed to be flattened along a certain length (200), the urethral cuff (1) wraps the urethra (100) more firmly but with a more homogeneous distribution of pressure thus preventing urinary incontinence more effectively while providing an enhanced patient compliance.

Moreover, when the urethral cuff (1) according to the present invention is implanted around the urethra (100) of a patient and all three inflatable cushions (10, 20, 30) are inflated, the urethra (100) is flattened at least in three different planes. These planes form certain angles with each other such that the flattened length (200) elongates with multiple breaking points and/or curved sections. This configuration allows the area of the flattened length (200) to be expanded, thus reducing the pressure applied per area even more while increasing the occlusion of the urethra (100). Furthermore, as the urethral cuff (100) is inflated around the urethra (100), all three inflatable cushions (10, 20, 30) inflate simultaneously, thus avoiding instantaneous local high pressure on the urethra (100).

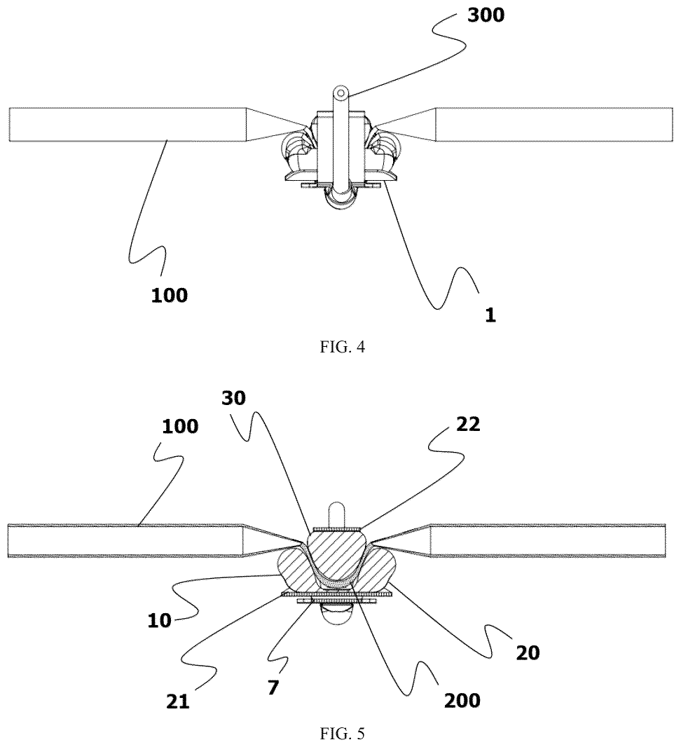

FIG. 4 shows a front view of a urethral cuff according to the present invention in a state as implanted around the urethra (100) and in its inflated state. This configuration enables the urethra (100) to narrow first from both sides approaching into the urethral cuff (1) and then to be completely flattened. Thus, the amount of the urethral fluid per length is reduced before it reaches the flattened length (200). This process also plays a role in ensuring continence. The flattened length (200) of the urethra (100) can be U-shaped as depicted in FIG. 5 or the length (200) can also be flattened so as to form a sinusoidal-wave shape.

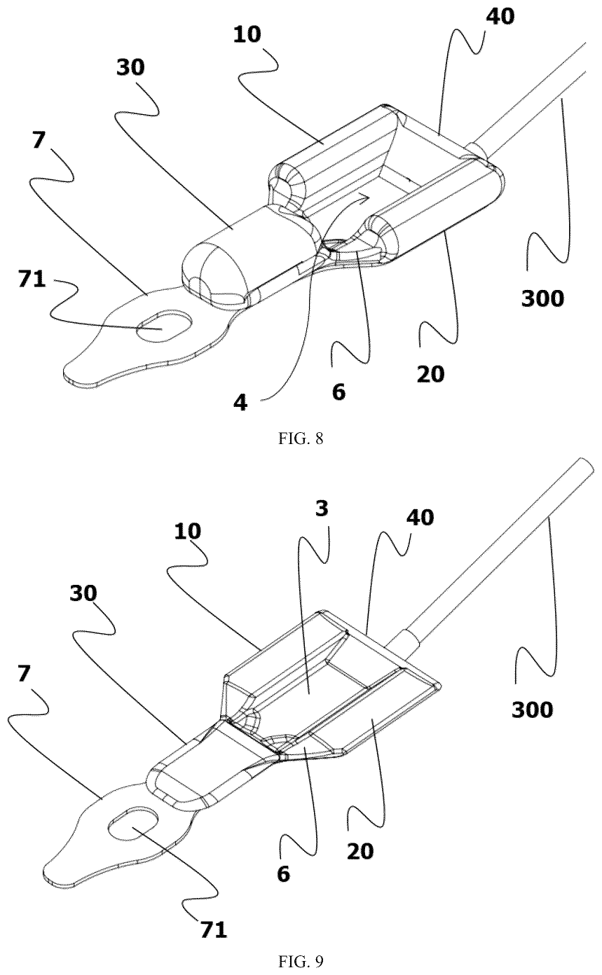

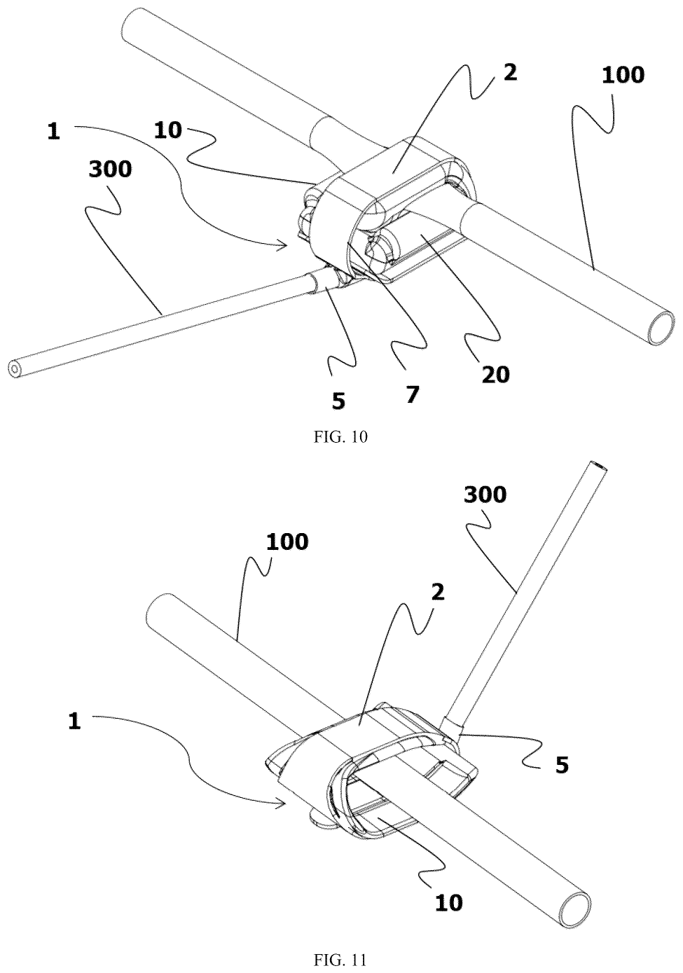

According to an embodiment, the first portion (21) further comprises a fourth inflatable cushion (40). The fourth inflatable cushion (40) connects the first and second inflatable cushions (10, 20) such that the two-dimensional gap (3) converts into a three-dimensional recess (4) when all four inflatable cushions (10, 20, 30, 40) are inflated. In this case, the third inflatable cushion (30) fits into and fills said recess (4) when the second portion (22) is folded on top of the first portion (21). As can be seen in FIG. 8, the recess (4) surrounded by the first, second and fourth inflatable cushions (10, 20, 40) is in the form of a housing in which the third inflatable cushion (30) can be fully seated. The third inflatable cushion (30) is sized and shaped to fit the recess (4) in the inflated state of the three cushions (10, 20, 30).

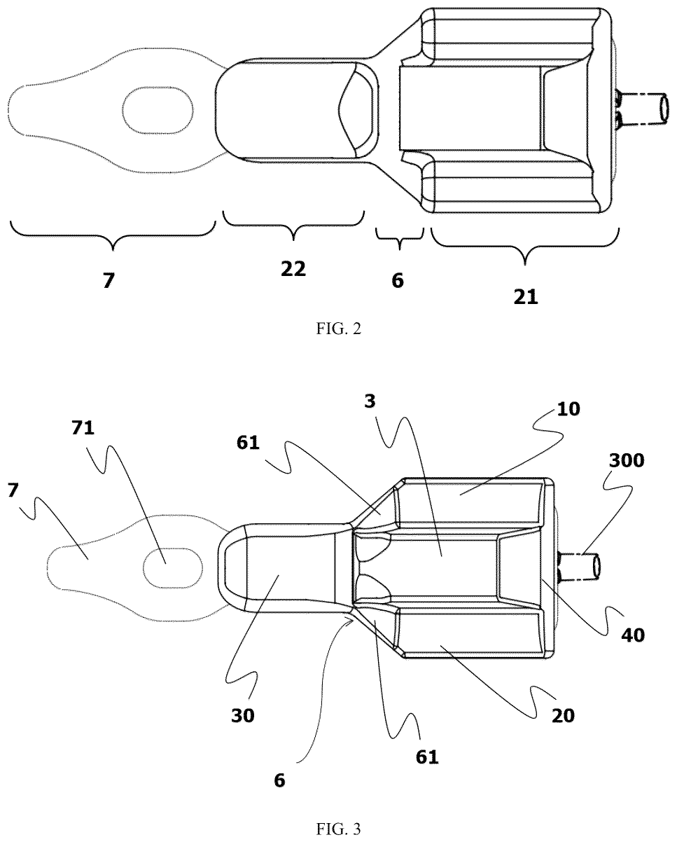

The urethral cuff (1) further comprises a folding section (6) that allows fluid communication between the first portion (21) and the second portion (22) by means of at least one conduit (61), thus allowing simultaneous inflation or deflation of all four inflatable cushions (10, 20, 30, 40). The folding section (6) is in the form of a bridge between the first portion (21) and the second portion (22), enabling the second portion (22) to be folded on top of the first portion (21) without creating an unintentional tension on the urethra (100). The folding section (6) is configured to be aligned with the third inflatable cushion (30) as it extends from the first portion (21) to the second portion (22) as depicted in FIG. 2 and FIG. 3. Said folding section (6) preferably contains two conduits (61), such that each conduit (61) is extending between the third inflatable cushion (30) and each of the first and second inflatable cushions (10, 20). Said conduits (61) ensure the simultaneous transfer of the pressurizing fluid to all three inflatable cushions (10, 20, 30), preferably to all four inflatable cushions (10, 20, 30, 40), regardless of where the pressurizing fluid enters into the urethral cuff (1).

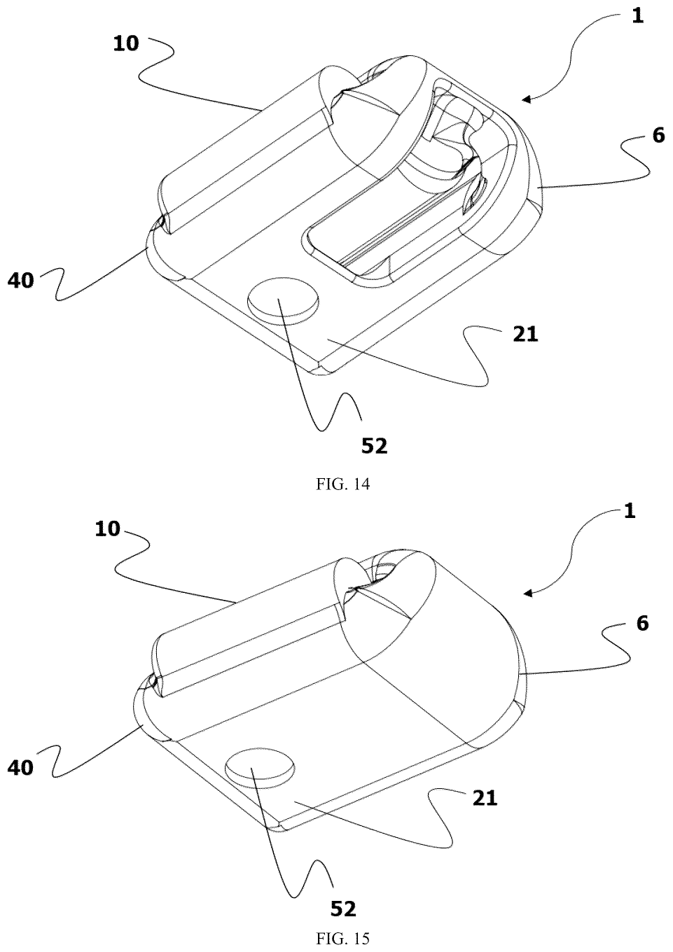

The backsheet (2) has an inlet (5) enabling the connection of a tube (300) for inflating all four inflatable cushions (10, 20, 30, 40) with a pressurizing fluid. Said inlet (5) is preferably located on the outer surface of the backsheet (2) such that the pressurizing fluid enters the urethral cuff (1) through the first portion (22). Accordingly, the pressurizing fluid coming from the tube (300) inflates the first and second inflatable cushions (10, 20) and preferably also the fourth inflatable cushion (40). Said pressurizing fluid is transferred to the third inflatable cushion (30) simultaneously via the conduits (61) in the folding section (6). Said inlet (5) is configured around an inlet hole (52) on the backsheet (2), preferably on the first portion (21) of the backsheet (2) as depicted in FIG. 14 and FIG. 15.

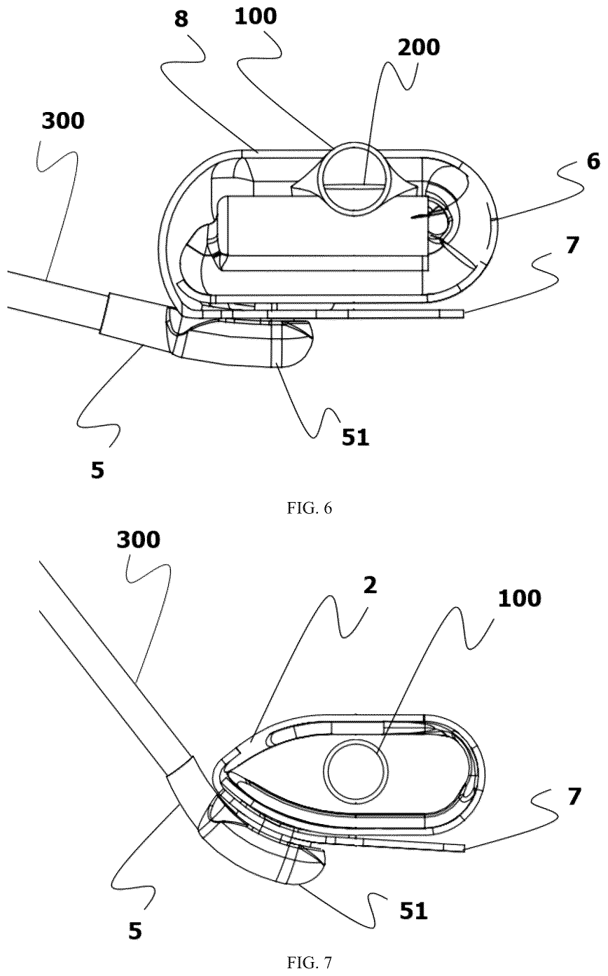

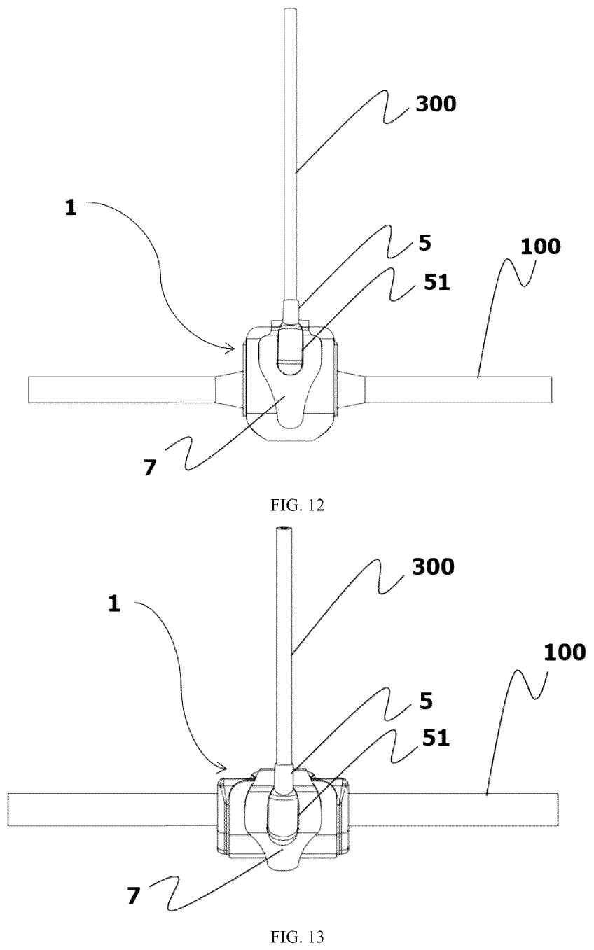

In an embodiment, the backsheet (2) is also provided with a pull tab (7). Said pull tab (7) comprises a connection hole (71) corresponding the inlet (5) when the second portion (22) is folded on top of the first portion (21). Accordingly, the inlet (5) is provided with a protrusion (51) for supporting the pull tab (7) through said hole (71). This configuration ensures that the pull tab (7) is locked around the inlet (5), when the urethral cuff (1) is implanted around the urethra (100) of a patient. Thus, the urethral cuff (1) is more securely fixed on the urethra (100). The top view of this locking mechanism can be seen in FIG. 12 and FIG. 13. Said protrusion (51) is in the form of a cap covering the inlet (5) and forming a cavity to ensure the attachment of the pull tab (7). FIG. 6 and FIG. 7 show the side views of the urethral cuff (1) in its inflated and deflated states, including said protrusion (51) structure.

According to an embodiment of the present invention, the backsheet (2) further has a reinforcement element (8) in the form of a mesh structure. Said reinforcement element (8) covers the backsheet (2) on its outer surface for enhancing strength and lifetime of the urethral cuff (1).

The present invention also proposes an artificial urinary sphincter comprising the urethral cuff (1) according to anyone of the embodiments described above.

According to an embodiment, said artificial urinary sphincter is provided with an electronic system which can be remotely controlled.

When used in an artificial urinary sphincter, said urethral cuff (1) is implanted around the urethra (100) of a patient and operated in coordination with a pump to which it is connected via the tube (300). In this case, said pump is operated by the patient or a physician for enabling the transmission of a pressurizing fluid from a fluid reservoir to said urethral cuff (1). The urethral cuff (1) is inflated as the pressurizing fluid moves into the urethral cuff (1) so as to occlude the urethra (100).

According to a use of the urethral cuff (1) as described by the present invention, when the patient needs to urinate, the patient shall operate the pump to transfer the fluid contained in the urethral cuff (1) into a fluid reservoir which is implanted in the abdomen of the patient and which has a stretchable wall. The pump preferably also prevents the fluid flow towards to the urethral cuff (1) by means of a check valve. As there is no pressurizing fluid in the urethral cuff (1), the patient is now free to urinate.

In the case of the inflation of the urethral cuff (1), the urethral cuff (1) is automatically inflated by the abdominal pressure of the patient as well as by the pressure increase arising from the stretching of the walls of the fluid reservoir. Since the fluid previously found in the urethral cuff (1) was transferred to the stretchable fluid reservoir, the internal pressure of the stretchable fluid reservoir is very high not only because the reservoir is in its stretched form but also because of the abdominal pressure of the patient. Inflation of the urethral cuff (1) occurs gradually because of the fact that the check valve allows a controlled reverse fluid flow towards the urethra (100). Once the controlled flow from the fluid reservoir inflates the urethral cuff (I), the artificial urinary sphincter reaches is stable form. The urethra (100) is now occluded and the patient is continent.

The urethral cuff (1) as described by the present invention overcomes incontinence without damaging the capillaries in the urethra and the corresponding tissue, thus reducing the possibility of atrophy or erosion. Another advantage of the present invention over the prior art is that the urethral cuff (1) according to anyone of the embodiments described above, when deflated, allows insertion of an endoscope or a resectoscope.

What is claimed is:

1. A urethral cuff for a use in an artificial urinary sphincter, the urethral cuff having a backsheet forming a body of the urethral cuff, wherein
the backsheet comprises a first portion and a second portion, the first portion has a first inflatable cushion and a second inflatable cushion separated from each other by a gap, the second portion has a third inflatable cushion;
the third inflatable cushion is located so as to correspond on a top of the gap when the second portion is folded on a top of the first portion such that when the urethral cuff is implanted around a urethra of a patient and the first inflatable cushion, the second inflatable cushion, and the third inflatable cushions are inflated, a length of the urethra is flattened among the first inflatable cushion, the second inflatable cushion, and the third inflatable cushion resulting in an occlusion of the urethra;
the first portion further comprises a fourth inflatable cushion connecting the first inflatable cushion and the second inflatable cushion such that the gap converts into a recess when the first inflatable cushion, the second inflatable cushion, the third inflatable cushion, and the fourth inflatable cushion are inflated;
the third inflatable cushion is sized and shaped to fit into and fill the recess when the second portion is folded on the top of the first portion and when the first inflatable cushion, the second inflatable cushion, the third inflatable cushion, and the fourth inflatable cushion are inflated.

2. (canceled)

3. The urethral cuff according to claim 1, wherein a flattened length forms a U-shaped longitudinal section.

4. The urethral cuff according to claim 1, wherein a flattened length forms a sinusoidal wave shaped longitudinal section.

5. The urethral cuff according to claim 1, wherein the urethral cuff further comprises a folding section allowing a fluid communication between the first portion and the second portion by at least one conduit such that the first inflatable cushion, the second inflatable cushion, and the third inflatable cushion are inflated simultaneously.

6. The urethral cuff according to claim 1, wherein the backsheet has an inlet enabling a transfer of a pressurizing fluid.

7. The urethral cuff according to claim 6, wherein the inlet is located on an outer surface of the backsheet.

8. The urethral cuff according to claim 6, wherein the backsheet is provided with a pull tab having a connection hole located such that the inlet corresponds into the connection hole when the second portion is folded on the top of the first portion.

9. The urethral cuff according to claim 8, wherein the inlet is provided with a protrusion for supporting the pull tab through the connection hole such that the pull tab is locked around the inlet upon an implantation of the urethral cuff around the urethra of the patient.

10. The urethral cuff according to claim 1, wherein the backsheet further comprises a reinforcement element in a form of a mesh structure.

11. (canceled)

12. An artificial urinary sphincter comprising the urethral cuff according to claim 1.

13. The urethral cuff according to claim 3, wherein the urethral cuff further comprises a folding section allowing a fluid communication between the first portion and the second portion by at least one conduit such that the first inflatable cushion, the second inflatable cushion, and the third inflatable cushion are inflated simultaneously.

14. The urethral cuff according to claim 4, wherein the urethral cuff further comprises a folding section allowing a fluid communication between the first portion and the second portion by at least one conduit such that the first inflatable cushion, the second inflatable cushion, and the third inflatable cushion are inflated simultaneously.

15. The urethral cuff according to claim 3, wherein the backsheet has an inlet enabling a transfer of a pressurizing fluid.

16. The urethral cuff according to claim 4, wherein the backsheet has an inlet enabling a transfer of a pressurizing fluid.

17. The urethral cuff according to claim 5, wherein the backsheet has an inlet enabling a transfer of a pressurizing fluid.

18. The urethral cuff according to claim 7, wherein the backsheet is provided with a pull tab having a connection hole located such that the inlet corresponds into the connection hole when the second portion is folded on the top of the first portion.

19. The urethral cuff according to claim 3, wherein the backsheet further comprises a reinforcement element in a form of a mesh structure.

20. The urethral cuff according to claim 4, wherein the backsheet further comprises a reinforcement element in a form of a mesh structure.

21. The urethral cuff according to claim 5, wherein the backsheet further comprises a reinforcement element in a form of a mesh structure.

22. The urethral cuff according to claim 6, wherein the backsheet further comprises a reinforcement element in a form of a mesh structure.
